(19) United States
(12) Patent Application Publication
(10) Pub. No.: US 2025/0031990 A1
(43) Pub. Date: Jan. 30, 2025

(54) METHOD AND DEVICE FOR DETERMINING AN AORTIC STATE

(71) Applicant: Technische Universität Graz, Graz (AT)

(72) Inventors: Sascha RANFTL, Graz (AT); Vahid BADELI, Graz (AT)

(21) Appl. No.: 18/714,507

(22) PCT Filed: Nov. 10, 2022
(86) PCT No.: PCT/EP2022/081506

§ 371 Date: May 29, 2024

(30) Foreign Application Priority Data
Dec. 2, 2021 (EP) 21212048.9

(51) Int. Cl. A61B 5/053; A61B 5/00; A61B 5/02

(52) U.S. Cl. CPC A61B 5/053; A61B 5/02007; A61B 5/6823; A61B 5/7242; A61B 5/7246; A61B 5/7271

(57) ABSTRACT

A method for determining an aortic state of a patient by a processing device having a processor and a memory, the method comprising the steps: receiving in said memory, from at least two sets of impedance cardiography (ICG) electrodes an impedance sensed over time; storing in said memory, for each of a set of aortic states and each electrode set, a surrogate model describing a reference impedance over time; computing, by means of the processor, for each aortic state of the set, a similarity value; and determining, by means of the processor, the aortic state with the highest computed similarity value as the patient's aortic state. The disclosed subject matter further relates to said processing device, and to a method and a measuring system for measuring the aortic state.

### CROSS-REFERENCE TO RELATED APPLICATION/S

This application is a National Phase application of International Application No. PCT/EP2022/081506 filed Nov. 10, 2022, which claims priority to the European Patent Application No. 21 212 048.9 filed Dec. 2, 2021, the disclosures of which are incorporated herein by reference in their entireties.

### TECHNICAL FIELD

The present disclosed subject matter relates to a method and a processing device for determining an aortic state of a patient. The disclosed subject matter further relates to a method and a measuring system for measuring the aortic state.

### BACKGROUND ART

Aortic pathologies, such as an aortic dissection, an aneurysm, an intramural hematoma, etc. are very serious and often even fatal when not treated properly and promptly. For proper treatment, the (pathological) aortic state has to be determined accurately. State of the art determination of whether a patient's aorta is in a healthy or in a (specific) pathologic state is performed on the basis of chest X-rays, computed tomography (CT), magnetic resonance imaging (MRI), which require expensive, large and bulky equipment, or on the basis of ultrasound imaging, the handling of which requires particular professional skills.

For more elementary measurements, e.g., of the heart rate or stroke volume, etc., impedance cardiography (ICG) has proven to be easy and reliable. In ICG, a pair of sensor electrodes is placed at specific spots on the patient's thorax defining a measuring segment therebetween; moreover, a pair of injector electrodes is placed at specific spots on the patient's thorax, each behind a different one of the sensor electrodes, outside the measuring segment. This configuration of a set of four electrodes is called a “tetra-polar” electrode configuration. Subsequently, an alternating current is injected along the thorax by means of the outer injector electrodes. A significant portion of the current passes through the aorta, and a respective voltage is captured by means of the inner sensor electrodes. The impedance is sensed over time by employing the known relation Z=U/I, wherein Z, U, and I denote the sensed impedance, the captured voltage, and the injected current, respectively. The sensed impedance, which can, e.g., be the absolute value of the impedance over one or more cardiac cycles, is subsequently analysed by a physician.

ICG electrodes can be easily placed on the patient's thorax such that the results of ICG measurements are quickly available. However, state of the art ICG measurements do not allow to accurately, reliably and easily determine the aortic state therefrom for the following reasons.

Firstly, it is particularly difficult per se to distinguish the sensed impedances of healthy aortas from pathologic aortas, not to mention a distinction between different pathologic states, e.g., between aortas showing an ascending aortic dissection, a descending aortic dissection, an aneurysm, an intramural haematoma, a penetrating atherosclerotic ulcer, or the like.

Secondly, in case of a pathologic aortic state, the sensitivity of the ICG electrodes strongly depends on the (unknown) position of a pathological change in the patient's aorta relative to the placement spots of the ICG electrodes. A low sensitivity to a pathological aortic change impedes a distinction between different aortic states. In the worst case, a pathological aortic change residing at an inauspicious position relative to the chosen spots of the ICG electrodes may not even significantly influence the impedance sensed over time and may, thus, result in a faulty assessment of the patient's aortic state and, consequently, even in a medical malpractice.

### BRIEF SUMMARY

It is an object of the present disclosed subject matter to provide a method and a processing device which can determine an aortic state of a patient, and a measuring system and method which can measure the aortic state of the patient, in each case reliably, accurately and automatically.

In a first aspect, this object is achieved with a method for determining an aortic state of a patient by means of a processing device which has a processor and a memory connected thereto, the method comprising:
receiving in said memory, from each of at least two different sets of impedance cardiography, ICG, electrodes placed in a tetra-polar electrode configuration at respective spots on the patient's thorax, an impedance sensed over time by that set of ICG electrodes;
prior to, during, or after said receiving, storing in said memory, for each of a predetermined set of aortic states and each set of ICG electrodes, a surrogate model generated for a plurality of reference objects having that aortic state, which surrogate model describes a reference impedance over time;
computing, by means of the processor, for each aortic state of the set, a similarity value from a product of probability functions each of which depends on a difference between the sensed impedance for a respective set of ICG electrodes and the reference impedance for that aortic state and the same set of ICG electrodes; and
determining, by means of the processor, the aortic state with the highest computed similarity value as the patient's aortic state.

The disclosed subject matter automatically combines impedances sensed over time by the different sets of ICG electrodes into one aortic state determination. By using at least two different sets of ICG electrodes in tetra-polar electrode configuration placed at different spots on the patient's thorax, inauspicious positions of potential pathological aortic changes of the patient are reliably precluded. Inaccuracies and incorrect analyses are greatly reduced or even eliminated. As applicant's research has shown, employing probability functions is particularly suited for efficiently computing the similarity values. An undue increase of computational complexity with the number of sensed impedances can be avoided.

The similarity values accurately indicate a similarity between the sensed and reference impedances and, thus, the respective aortic state. Moreover, the similarity values are not only suitable to determine the aortic state but also—when comparing their magnitudes—to estimate the certainty of the determined aortic state. For example, when the difference of similarity values of a healthy state, which has been determined as the patient's aortic state, and a pathologic state is rather small, precautionary measures such as a further measurement may be taken.

The probability function may be a distribution function such as a log-normal distribution, a Pareto distribution, a gamma distribution, a Student's t-distribution, etc., each with its maximum corresponding to said difference between the sensed and reference impedances being zero.

In a favourable embodiment of the disclosed subject matter, each probability function is a Gaussian function according to

$$\frac{1}{\sqrt{2 π {Δ}_{i , j , k}^{2}}} exp ( - \frac{{( {Z}_{j} ( {t}_{k} ) - {f}_{i , j} ( {t}_{k} ) )}^{2}}{2 {Δ}_{i , j , k}^{2}} ) \qquad ( 1 )$$

with
exp . . . the exponential function,
Zⱼ(tₖ) . . . the sensed impedance for the j^{th} set of ICG electrodes at the k^{th} sensing time,
f_{i,j}(tₖ) . . . the reference impedance for the i^{th} aortic state and the j^{th} set of ICG electrodes at the k^{th} sensing time, and
Δ_{i,j,k} . . . an estimated uncertainty as to the sensing by the j^{th} set of ICG electrodes and/or as to the reference impedance for the i^{th} aortic state and the j^{th} set of ICG electrodes at the k^{th} sensing time.

The Gaussian function accounts for the estimated uncertainty with regard to the sensing and/or with regard to the reference impedance in a simple manner, which further increases the accuracy of the determination of the aortic state of the patient. Moreover, the product of exponential functions is particularly efficiently computed by the processor, e.g., by summing the exponents and evaluating a resulting single exponential function.

In an optional embodiment of the disclosed subject matter, each reference impedance is an expansion of basis functions which depend on predefined aortic parameters of the aortic state, and said step of computing is performed by marginalising the product of probability functions with respect to the aortic parameters. Such an expansion takes aortic parameters into account, e.g., an outward bulging of parts of the aorta, mechanical parameters, e.g., a stiffness, of the aorta, a blood composition, or a position of a pathologic change, and, thus, renders the similarity value computation particularly accurate. While the values of these aortic parameters can be determined, e.g., measured, for the reference objects when generating the surrogate models, the aortic parameters of the patient need not be determined in separate time-consuming measurements. This is due to said marginalising, wherein each similarity value is computed as a marginal distribution by integrating, over the aortic parameters, the product of probability functions multiplied by a predetermined prior probability for the aortic parameters and for the aortic state. The prior probability, which indicates the probability for the values of the aortic parameters being present given the aortic state, may be accurately measured or efficiently estimated.

Advantageously, the aortic parameters of a healthy aortic state are a haematocrit level and a maximum radius of the true lumen. Favourably, the aortic parameters of a dissected aortic state are a maximum radius of the true lumen, a maximum radius of the false lumen, a haematocrit level, and a position of the false lumen relative to the true lumen. As applicant's research has shown, these aortic parameters can be significant for accurately describing the reference impedances of reference objects with a healthy and a pathologic aorta, respectively.

It is beneficial when, in said step of computing, each similarity value is computed according to

$$p ( {AS}_{i} \vert Z ) = \frac{p ( {AS}_{i} )}{Vol ( {R}_{i} )} {\int }_{{R}_{i}} p ( Z \vert {x}_{i} , {AS}_{i} ) d {V}_{{x}_{i}} \qquad ( 2 )$$

optionally according to

$$p ( {AS}_{i} \vert Z ) = \frac{p ( {AS}_{i} )}{V o l ( {R}_{i} )} {\int }_{{R}_{i}} {\prod }_{{j = 1 , … , J}_{k = 1 , … , K}} \frac{1}{\sqrt{2 π {Δ}_{i , j , k}^{2}}} exp ( - \frac{{( {Z}_{j} ( {t}_{k} ) - {f}_{i , j} ( {x}_{i} , {t}_{k} ) )}^{2}}{2 {Δ}_{i , j , k}^{2}} ) {dV}_{{x}_{i}} \qquad ( 3 )$$

with
p(ASᵢ|Z) . . . the computed similarity value of the i^{th} aortic state given the sensed impedances Z,
p(ASᵢ) . . . a predetermined prior probability for the i^{th} aortic state,
Rᵢ . . . a predetermined set of values of the aortic parameters xᵢ of the i^{th} aortic state,
Vol(Rᵢ) . . . a hypervolume spanned by said predetermined set of values,
p(Z|xᵢ,ASᵢ) . . . the product of probability functions,
dVₓ_{ᵢ} . . . a hypervolume element,

$${\int }_{{R}_{i}}$$

an integration over the predetermined set of values,

$${\prod }_{{j = 1 , … , J}_{k = 1 , … , K}}$$

a product over all J sets of ICG electrodes and all K sensing times,
Zⱼ(tₖ) . . . the sensed impedance for the j^{th} set of ICG electrodes at the k^{th} sensing time,
f_{i,j}(xᵢ,tₖ) . . . the reference impedance for the i^{th} aortic state and the j^{th} set of ICG electrodes at the k^{th} sensing time in dependence on the aortic parameters xᵢ of the i^{th} aortic state, and
Δ_{i,j,k} . . . an estimated uncertainty as to the sensing by the j^{th} set of ICG electrodes and/or as to the reference impedance for the i^{th} aortic state and the j^{th} set of ICG electrodes at the k^{th} sensing time.

In this case, the marginalising is a particularly simple and efficient computation, which reduces the required computing power and/or time. In addition, this marginalising does not require a complex predetermination of a prior probability of the aortic parameters for the respective aortic state.

Advantageously, in said step of computing, each reference impedance is calculated by means of the processor from the surrogate model which describes the reference impedance by

$${f}_{i , j} ( {x}_{i} , {t}_{k} ) = {f}_{i , j} ( {x}_{i} ; {c}_{i} ( {t}_{k} ) ) = {\sum }_{q = 1 , … , Q} {c}_{i , j , q} ( {t}_{k} ) \cdot {ϕ}_{q} ( {x}_{i} ) \qquad ( 4 )$$

with
f_{i,j}(xᵢ,tₖ) . . . the calculated reference impedance for the i^{th} aortic state and the j^{th} set of ICG electrodes at the k^{th} sensing time in dependence on the aortic parameters xᵢ of the i^{th} aortic state,
ϕ_{q}(xᵢ) . . . a multi-variate polynomial in the aortic parameters xᵢ of the i^{th} aortic state, wherein the polynomials are indexed by q,
c_{i,j,q}(tₖ) . . . an expansion coefficient for the i^{th} aortic state, the j^{th} set of ICG electrodes and the q^{th} polynomial at the k^{th} sensing time,
cᵢ . . . the set of all expansion coefficients for the i^{th} aortic state, and

$${\sum }_{q = 1 , … , Q}$$

a sum over all Q multi-variate polynomials.

With this expansion, a particularly accurate and fast measurement of the aortic state is achieved. Moreover, such an expansion in multi-variate polynomial basis functions is particularly simple and allows to calculate the reference impedances quickly and efficiently and, thus, requires only little processor resources.

In some embodiments, the surrogate models are generated in a step preceding the above-mentioned method, e.g. from real world reference objects such as humans or animals. In a favourable variant, however, the reference objects are finite element method, FEM, models of thoraces and the method further comprises, prior to said step of storing, generating, by means of the processor, each surrogate model by:
determining, for each of the reference objects, the values of the aortic parameters of the aortic state of the reference object;
applying, for each set of ICG electrodes and each of the reference objects, the ICG electrodes on that reference object's thorax at the respective spots and quantifying, by means of the applied ICG electrodes, an impedance over time; and
calculating, by means of the processor from the determined values of the aortic parameters and the quantified impedances, the expansion coefficients.

In this variant, the steps of determining, applying and quantifying can be carried out by creating the FEM model thorax with desired values of the aortic parameters and simulating the resulting impedance over time for the respective spots of the ICG electrodes of each set. This can be done in a fast and accurate manner, which allows to quickly create a large set of reference objects for a desired range of aortic parameters and the respective quantified impedances.

The expansion coefficients may be calculated by the processor by regression or by fitting the surrogate models to the respective quantified impedances of the same set of ICG electrodes and the same aortic state. However, it is particularly beneficial when the expansion coefficients are calculated according to

$${c}_{i , j , q} ( {t}_{k} ) = {\sum }_{l} {( {( {U}_{i}^{T} \cdot {U}_{i} )}^{- 1} \cdot {U}_{i}^{T} )}_{ql} {Z}_{j}^{l} ( {t}_{k} ) \qquad ( 5 )$$

with
c_{i,j,q}(tₖ) . . . the expansion coefficient for the i^{th} aortic state, the j^{th} set of ICG electrodes and the q^{th} polynomial at the k^{th} sensing time,
Uᵢ=ϕ_{q}(vᵢ^{l}) . . . a matrix for the i^{th} aortic state formed by evaluating the q^{th} multi-variate polynomial at the determined values vᵢ^{l} of the aortic parameters xᵢ of the l^{th} reference object having the i^{th} aortic state, and
Zⱼ^{l}(tₖ) . . . the quantified impedance of the l^{th} reference object in the j^{th} set of ICG electrodes at the k^{th} sensing time.

Surrogate models generated with these calculated expansion coefficients describe reference impedances which, on average, have the smallest error with respect to the underlying quantified impedances of the reference objects and, consequently, facilitate a particularly accurate determination of the aortic state.

The impedances can be sensed in preliminary steps before the above-mentioned method is carried out and provided to the processing device for determination of the aortic state. In a second aspect, however, the disclosed subject matter provides for a method for measuring an aortic state of a patient, which method comprises the abovementioned method for determining the aortic state and, prior to said step of receiving, for each set of ICG electrodes:
placing the ICG electrodes of that set in the tetra-polar electrode configuration at respective spots on the patient's thorax,
sensing, by means of that set of ICG electrodes, the respective impedance over time, and
transmitting, by means of that set of ICG electrodes, the sensed impedance to the memory.

This method of measuring the aortic state of the patient is uncoupled from preceding sensing steps carried out on the patient's thorax and is, thus, self-contained.

In a third aspect, the disclosed subject matter provides for a processing device for determining an aortic state of a patient, the processing device having
a memory which is configured to receive, from at least two different sets of impedance cardiography, ICG, electrodes placed in a tetra-polar electrode configuration at respective spots on the patient's thorax, an impedance sensed over time by that set of ICG electrodes, and to store, for each of a predetermined set of aortic states and each set of ICG electrodes, a surrogate model generated for a plurality of reference objects having that aortic state, which surrogate model describes a reference impedance over time; and
a processor connected to said memory and configured to compute, for each aortic state of the set, a similarity value from a product of probability functions each of which depends on a difference between the sensed impedance for a respective set of ICG electrodes and the reference impedance for that aortic state and the same set of ICG electrodes, and to determine the aortic state with the highest computed similarity value as the patient's aortic state.

With respect to the advantages and variants of the processing device reference is made to the abovementioned embodiments and variants of the method.

In a fourth aspect, the disclosed subject matter provides for a measuring system comprising the above-mentioned processing device and said at least two different sets of ICG electrodes, wherein each set of ICG electrodes is configured to be placed in the tetra-polar electrode configuration at the respective spots on the patient's thorax, to sense said impedance over time, and to transmit the sensed impedance to the memory.

Relating to further embodiments and variants of the measuring system and the advantages thereof, reference is made to the above statements on the method.

### BRIEF DESCRIPTION OF THE DRAWINGS/FIGURES

The disclosed subject matter shall now be described in further detail by means of exemplary embodiments thereof under reference to the enclosed drawings, in which:

FIG. 1 shows a measuring system according to the disclosed subject matter for measuring an aortic state of a patient in a schematic view;

FIG. 2 shows a method according to the disclosed subject matter carried out by the measuring system of FIG. 1 in a flow diagram;

FIG. 3 shows an impedance sensed over time by a set of impedance cardiography electrodes of the measuring system of FIG. 1 in a diagram over time;

FIG. 4 shows optional steps of a variant of the method of FIG. 2 in a flow diagram; and

FIG. 5 shows a finite element method (FEM) model of a thorax employed in the variant of FIG. 4 in an oblique perspective view from behind.

### DETAILED DESCRIPTION

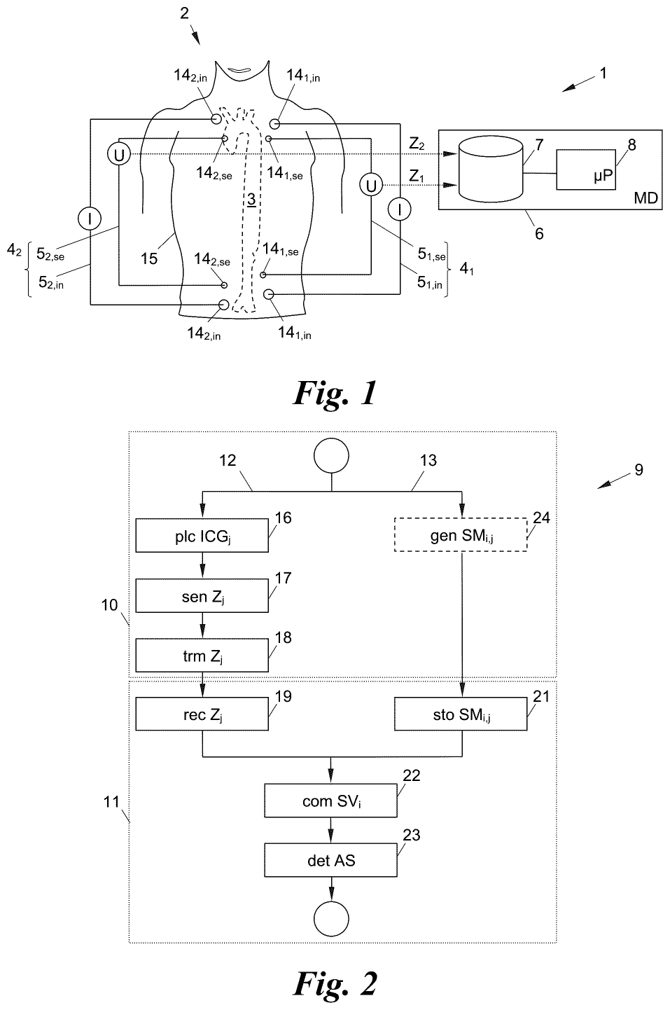

FIG. 1 shows a measuring system 1 which measures an aortic state of a patient 2. The patient 2 may be human or animal and the state of the patient's aorta 3 is one of a predetermined set of two or more aortic states. The set contains a healthy state and one or more pathologic states, e.g., a state of an ascending aortic dissection, a state of a descending aortic dissection, an aneurysmal state, etc., as required.

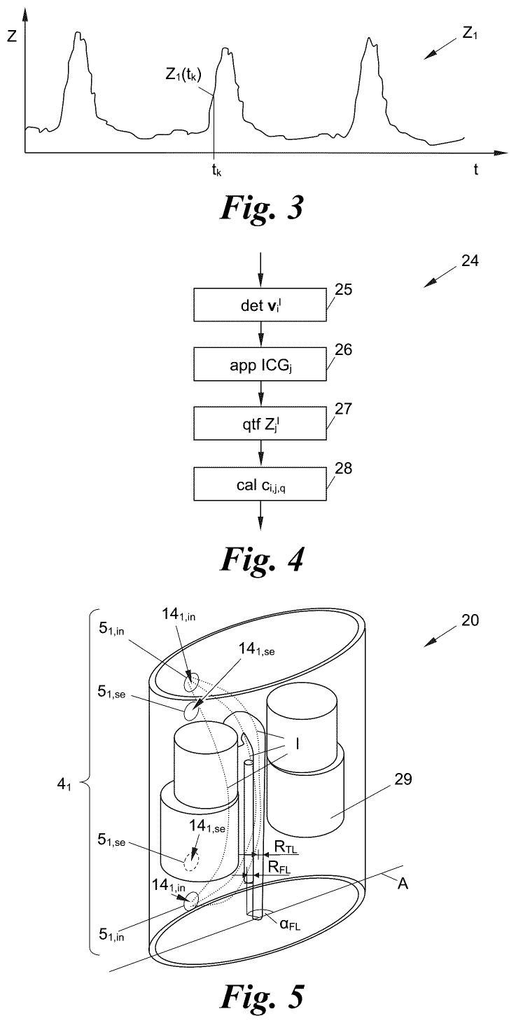

To measure the aortic state of the patient 2, the measuring system 1 comprises at least two different sets 4₁, 4₂, . . . 4ⱼ, generally 4ⱼ, of impedance cardiography (ICG) electrodes 5_{1,in}, 5_{1,se}, 5_{2,in}, 5_{2,se}, . . . , 5_{J,in}, 5_{J,se}, generally 5_{j,in}, 5_{j,se}, for sensing a respective impedance Z₁, Z₂, . . . Z_{J}, generally Zⱼ, over time (FIG. 3). The measuring system 1 also comprises a processing device 6 which determines the aortic state of the patient 2 based on the impedances Zⱼ sensed over time by the ICG electrodes 5_{j,in}, 5_{j,se}, respectively. To this end, the processing device 6 has a memory 7 and a processor 8 connected to the memory 7.

With reference to FIGS. 1 to 3, a method 9 carried out by the measuring system 1 for measuring the aortic state shall now be described.

As illustrated in FIG. 2, the method 9 is divided into two phases 10, 11, wherein the first phase 10 substantially comprises the sensing of the impedances Zⱼ (branch 12). Surrogate models are provided and optionally generated in a branch 13, either in the first or in the second phase 10, 11 (here: in the first phase 10). The second phase 11 is directed at determining the patient's aortic state based on the sensed impedances Zⱼ and the surrogate models. Taken in isolation, this phase 11 of determining constitutes a method on its own which is carried out by the processing device 6.

During the first phase 10 in the first branch 12, the ICG electrodes 5_{j,in}, 5_{j,se} of each set 4ⱼ are placed in a tetra-polar electrode configuration at respective spots 14_{j,in}, 14_{j,se} on the patient's thorax 15 in step 16. To this end, each set 4ⱼ of ICG electrodes 5_{j,in}, 5_{j,se} (herein also: “electrode set” 4ⱼ) comprises a pair of sensor electrodes 5_{j,se} which is placed at specific sensing spots 14_{j,se} on the patient's thorax 15, forming a measuring segment therebetween, and a pair of injector electrodes 5_{j,in} which is placed at specific injecting spots 14_{j,in} on the patient's thorax 15, each behind a different one of the sensor electrodes 5_{j,se}, outside the measuring segment. The specific spots 14_{j,in}, 14_{j,se} are predetermined for each set 4ⱼ of ICG electrodes 5_{j,in}, 5_{j,se} and may be at the front, the side and/or the back of the thorax 15. It shall be mentioned that, for the sake of easier understanding, two sets 4₁, 4₂ of a total of eight ICG electrodes 5_{1,in}, 5_{1,se}, 5_{2,in}, 5_{2,se} are identified in FIG. 1; however, the shown ICG electrodes 5_{1,in}, 5_{1,se}, 5_{2,in}, 5_{2,se} can be deployed to form further electrode sets, e.g., an additional electrode set formed by the right injector electrodes 5_{1,in} and the left sensor electrodes 5_{2,se}, or an additional electrode set formed by the respective upper ones of the left injector electrodes 5_{2,in} and of the left sensor electrodes 5_{2,se} and by the respective lower ones of the right injector electrodes 5_{1,in} and of the right sensor electrodes 5_{1,se}, etc. Moreover, further sensor and/or injector electrodes 5_{j,in}, 5_{j,se} forming one or more additional electrode sets 4ⱼ may be placed on the patient's thorax 15. Generally speaking, any tetra-polar configuration of sensor and injector electrodes 5_{j,in}, 5_{j,se} may be employed to form the electrode sets 4ⱼ. Hence, an electrode set 4ⱼ may optionally even comprise more than two injector electrodes.

In step 17, a respective impedance Zⱼ is sensed over time for each electrode set 4ⱼ by the ICG electrodes 5_{j,in}, 5_{j,se} of that electrode set 4ⱼ. This is achieved by injecting an alternating current I into the thorax 15 by means of the injector electrodes 5_{j,in} of the electrode set 4ⱼ and by sensing a resulting voltage U and, together therewith, the impedance Zⱼ using the relation Zⱼ=U/I by means of the sensor electrodes 5_{j,se} of the electrode set 4ⱼ.

In the present method 9, the impedance Zⱼ is either a complex value taking into account the phase between the current I and the voltage U, the modulus thereof, and/or the derivative of one of the two.

FIG. 3 shows an example for an impedance Z₁ that has been sensed over time (here: its modulus) by means of the first set 4₁ of ICG electrodes 5_{1,inj}, 5_{1,sens} over three cardiac cycles; at an exemplary sensing time tₖ, the sensed impedance Z₁ is Z₁(tₖ).

Steps 16 and 17 may be carried out one after the other, i.e., by placing the ICG electrodes 5_{j,in}, 5_{j,se} of all electrode sets 4ⱼ on the patient's thorax 15 and then sensing the respective impedances Zⱼ over time one after the other or even simultaneously, e.g., at respective different frequencies; alternatively, steps 16 and 17 may be intermeshed in that the ICG electrodes 5_{1,in}, 5_{1,se} of a single electrode set 4₁ and 4₂, respectively, are initially placed at the spots 14_{1,in}, 14_{1,se} on the thorax 15 forming the first electrode set 4₁, and the first impedance Z₁ is then sensed over time, thereafter the same ICG electrodes 5_{1,in}, 5_{1,se} are placed at different spots 14_{2,in}, 14_{2,se} on the thorax 15, forming the second electrode set 4₂, and the second impedance Z₂ is sensed over time (not shown), etc.

Coming back to FIG. 2, in step 18, for each set 4ⱼ of ICG electrodes, the impedance Zⱼ sensed by the electrode set 4ⱼ is transmitted by means of the electrode set 4ⱼ to the memory 7, either one after the other or as a packet of two or more impedances Z₁, Z₂, . . . , e.g., via a wired or a wireless connection as known in the art.

In final step 19 of the first branch 12 which constitutes the starting point of the second phase (or method) 11, the respective transmitted impedance Zⱼ is received in memory 7 from each of the at least two different electrode sets 4ⱼ.

In the second branch 13, the processing device 6 is provided with a respective surrogate model for each of the predetermined sets of aortic states and for each set 4ⱼ of ICG electrodes 5_{j,in}, 5_{j,se}. Each surrogate model is generated for a plurality of reference objects 20 (FIG. 5) having that aortic state and describes a reference impedance over time. Each reference impedance over time, thus, corresponds to a set 4ⱼ of ICG electrodes 5_{j,in}, 5_{j,se} placed at the respective abovementioned spots 14_{j,in}, 14_{j,se}.

The generation of the surrogate models will be explained in detail with respect to FIGS. 4 and 5 further down.

In step 21 which is part of the second phase 11, the generated surrogate models are stored in the memory 7 and, thus, are accessible to the processor 8. Each surrogate model is stored in such a format that the processor 8 can determine the described reference impedance over time therefrom, e.g., as a parameterised function, as the reference impedances for respective sensing times tₖ in an array, or the like.

When the first and second branches 12, 13 have been passed, the processor 8 determines the aortic state of the patient 2 from the sensed impedances Zⱼ and the reference impedances. To this end, the processor 8 computes, for each aortic state, a similarity value from a product of probability functions in step 22. In one variant, the similarity value is computed by multiplying the product with a predetermined prior probability for the respective aortic state; in another variant, which is described below, a more elaborate method is performed. Each probability function depends on a difference between the impedance Zⱼ sensed by one set 4ⱼ of ICG electrodes 5_{j,in}, 5_{j,se} and the reference impedance for that aortic state and the same set 4ⱼ of ICG electrodes 5_{j,in}, 5_{j,se}, i.e., the ICG electrodes 5_{j,in}, 5_{j,se} placed (or applied, as described further below) at equivalent spots 14_{j,in}, 14_{j,se} on the thorax 15 of the patient 2 and on the thorax of the reference objects 20, respectively. Consequently, each of the similarity values computed in step 22 is indicative of a similarity between the sensed impedances Zⱼ and the reference impedances described by the surrogate model for the respective aortic state.

In a first variant of step 22, each probability function is taken at the respective sensing time t₁, t₂, . . . , tₖ, generally tₖ, and depends on said difference according to

$$F {P}_{i , j} = F {P}_{i , j} ( {t}_{k} ) = F {P}_{i , j} ( {Z}_{j} ( {t}_{k} ) - {f}_{i , j} ( {t}_{k} ) ) \qquad ( 6 a )$$

and the product is calculated as

$$p ( Z \vert {AS}_{i} ) = {{\prod }_{j = 1 , … , J}}_{k = 1 , … , K} {FP}_{i , j} ( {Z}_{j} ( {t}_{k} ) - {f}_{i , j} ( {t}_{k} ) ) . \qquad ( 6 b )$$

with
p(Z|ASᵢ) . . . the product of probability functions calculated from all sensed impedances Z={Z₁(t), Z₂(t), . . . , Z_{J}(t)} for the i^{th} aortic state ASᵢ (bold printing indicating a set),

$${{\prod }_{j = 1 , … , J}}_{k = 1 , … , K}$$

a product over all J sets of ICG electrodes and all K sensing times,
FP_{i,j} . . . the probability function for the i^{th} aortic state ASᵢ and the j^{th} set 4ⱼ of ICG electrodes 5_{j,in}, 5_{j,se},
Zⱼ(tₖ) . . . the sensed impedance for the j^{th} set 4ⱼ of ICG electrodes 5_{j,in}, 5_{j,se} at the k^{th} sensing time,
f_{i,j}(tₖ) . . . the reference impedance for the i^{th} aortic state ASᵢ and the j^{th} set 4ⱼ of ICG electrodes 5_{j,in}, 5_{j,se} at the k^{th} sensing time tₖ.

In a second variant of step 21, each probability function is taken for a respective time interval Δtₖ=tₖ₊₁−tₖ, and depends on said difference, e.g., according to

$$F {P}_{i , j} = F {P}_{i , j} ( Δ {t}_{k} ) = F {P}_{i , j} ( {\int }_{{t}_{k}}^{{t}_{k + 1}} ( {Z}_{j} ( t ) - {f}_{i , j} ( t ) ) d t ) , \qquad ( 7 a )$$

with
∫dt . . . an integration over time,
and the product is calculated as

$$p ( Z \vert {AS}_{i} ) = {{\prod }_{j = 1 , … , J}}_{k = 1 , … , K - 1} {FP}_{i , j} ( {\int }_{{t}_{k}}^{{t}_{k + 1}} ( {Z}_{j} ( t ) - {f}_{i , j} ( t ) ) dt ) . \qquad ( 7 b )$$

The sizes of the time intervals Δtₖ may, e.g., be small (in the range of several μs), medium (in the range of several ms), or even large (in the range of, e.g., a cardiac cycle or of the whole sensing time). In further variants of this embodiment, instead of the integral employed in equations (7a) and (7b), another mathematical functional known in the art can be employed to map the difference over time to a scalar.

In further variants, the product is calculated as a weighted product, e.g., as

$$p ( Z \vert {AS}_{i} ) = {\prod }_{j , k} F {P}_{i , j} ( {Z}_{j} ( {t}_{k} ) - {f}_{i , j} ( {t}_{k} ) ) \cdot {w}_{j , k} \qquad ( 8 )$$

wherein at least one weight w_{j,k} for the j^{th} set 4ⱼ of ICG electrodes and the k^{th} sensing time tₖ has a value not equal to one, e.g., to take correlations into account.

Subsequent to step 22, in final step 23 of the method 9, the processor 8 determines the aortic state with the highest computed similarity value as the patient's aortic state. Therefor, the processor 8 may simply compare the computed similarity values. The determined patient's aortic state may then be output, e.g., on a display (not shown). Optionally, all computed similarity values may be output, e.g., to yield an indication for the certainty of each determined aortic state for reason of comparison.

Below, embodiments and variants of the step 22 of computing the similarity values shall be described.

Each probability function employed in step 22 is, e.g., a function having larger values the smaller the difference between the sensed and the reference impedances of the respective aortic state, i.e., between Zⱼ and f_{i,j}, is. In an optional embodiment, each probability function is a Gaussian function according to

$$F {P}_{i , j} ( {t}_{k} ) = \frac{1}{\sqrt{2 {πΔ}_{i , j , k}^{2}}} exp ( - \frac{{( {Z}_{j} ( {t}_{k} ) - {f}_{i , j} ( {t}_{k} ) )}^{2}}{2 {Δ}_{i , j , k}^{2}} ) \qquad ( 1 )$$

with
FP_{i,j}(tₖ) . . . the probability function for the i^{th} aortic state and the j^{th} set of ICG electrodes 5_{j,in}, 5_{j,se} at the k^{th} sensing time tₖ,
exp . . . the exponential function, and
Δ_{i,j,k} . . . an estimated uncertainty as to the sensing by the j^{th} set 4ⱼ of ICG electrodes 5_{j,in}, 5_{j,se} and/or as to the reference impedance f_{i,j} for the i^{th} aortic state and the j^{th} set 4ⱼ of ICG electrodes 5_{j,in}, 5_{j,se} at the k^{th} sensing time tₖ.

In alternative embodiments, the probability function may, e.g., be a log-normal distribution, a Pareto distribution, a gamma distribution, a Student's t-distribution, etc. with the respective maximum corresponding to the difference between the sensed and reference impedances being zero. In further alternative embodiments, correlations between the sensed impedances Zⱼ may be considered, e.g., by extending the exponent of equation (1) with a covariance function depending on two or more sensed impedances Zⱼ.

In a further optional embodiment, which may be combined with the other embodiments, each reference impedance is an expansion of basis functions which depend on predefined aortic parameters of the aortic state. Therein, the product of probability functions also depends on the aortic parameters, i.e., p(Z|ASᵢ)=p(Z|xᵢ,ASᵢ) wherein xᵢ denotes the aortic parameters, and step 22 is performed by marginalising the product with respect to the aortic parameters.

The procedure of marginalising is known from statistics, in particular from Bayesian statistics, and is carried out by first multiplying the product (comparable to a likelihood in Bayesian statistics) with a predetermined prior probability for the aortic parameters given the aortic state and a predetermined prior probability for the aortic state, and then integrating the result over the aortic parameters. Thereby, each similarity value is computed in a statistical manner, also taking the aortic parameters into account. To this end, the values of the aortic parameters are determined when generating the surrogate models but do not have to be determined for the patient 2.

The aortic parameters are, in general, selected as parameters which allow for a distinction between the aortic states. For example, the aortic parameters of a healthy aortic state may be a haematocrit level and a maximum radius of the true lumen R_{TL} (FIG. 5), i.e., xᵢ={L_{H}, R_{TL}}, and/or the aortic parameters of a dissected aortic state may be a haematocrit level L_{H}, a maximum radius of the true lumen R_{TL}, a maximum radius of the false lumen R_{FL} (FIG. 5), and a position of the false lumen relative to the true lumen (FIG. 5: α_{FL}), i.e., x₂={L_{H}, R_{TL}, R_{FL}, α_{FL}}.

In an optional variant, each similarity value is computed according to

$$p ( {AS}_{i} \vert Z ) = \frac{p ( {AS}_{i} )}{V ol ( {R}_{i} )} {\int }_{{R}_{i}} p ( Z \vert {x}_{i} , {AS}_{i} ) d {V}_{{x}_{i}} \qquad ( 2 )$$

in particular according to

$$p ( {AS}_{i} \vert Z ) = \frac{p ( {AS}_{i} )}{Vol ( {R}_{i} )} {\int }_{{R}_{i}} {{\prod }_{j = 1 , … , J}}_{k = 1 , … , K} \frac{1}{\sqrt{2 {πΔ}_{i , j , k}^{2}}} exp ( - \frac{{( {Z}_{j} ( {t}_{k} ) - {f}_{i , j} ( {x}_{i} , {t}_{k} ) )}^{2}}{2 {Δ}_{i , j , k}^{2}} ) d {V}_{{x}_{i}} \qquad ( 3 )$$

with
p(ASᵢ|Z) . . . the similarity value of the i^{th} aortic state ASᵢ given the sensed impedances Z,
p(ASᵢ) . . . a predetermined prior probability for the i^{th} aortic state ASᵢ,
Rᵢ . . . a predetermined set of values of the aortic parameters xᵢ of the i^{th} aortic state ASᵢ,
Vol(Rᵢ) . . . a hypervolume spanned by said predetermined set of values,
dVₓ_{ᵢ} . . . a hypervolume element,

$${\int }_{{R}_{i}}$$

an integration over the predetermined set of values, and
f_{i,j}(xᵢ,tₖ) . . . the reference impedance for the i^{th} aortic state ASᵢ and the j^{th} set 4ⱼ of ICG electrodes 5_{j,in}, 5_{j,se} at the k^{th} sensing time tₖ in dependence on the aortic parameters xᵢ of the i^{th} aortic state ASᵢ.

The so computed similarity value is comparable to a posterior probability of the aortic state given the sensed impedances Zⱼ, and the product of probability functions is comparable to a (joint) likelihood (in the sense of Bayesian inference) which is marginalised via an uninformative uniform prior probability for the aortic parameters and multiplied by a predetermined prior probability for the aortic state. Thereby, the integration is restricted to the predetermined set of values of the respective aortic parameters. The set of values is a (often multidimensional) interval, for which the generated surrogate models of the respective aortic state yield meaningful results.

The afore-mentioned hypervolume is confined by the set of values Rᵢ and is one-dimensional in case of a single aortic parameter, e.g., xᵢ={L_{H}}, two-dimensional in case of two aortic parameters, e.g., xᵢ={R_{TL}, R_{FL}}, and so forth. As an example, the maximum radius of the true lumen R_{TL} may lie between 1 cm and 2.2 cm, optionally between 1.35 cm and 1.95 cm, and the maximum radius of the false lumen R_{FL} between 0.1 cm and 2 cm, optionally between 0.3 cm and 1.5 cm for the healthy state, resulting in a set of values R₁={[1.35 cm; 1.95 cm], [0.3 cm; 1.5 cm]} for the healthy state and a hypervolume Vol(Rᵢ)=(1.95-1.35) cm×(1.5-0.3) cm=0.72 cm². In other examples, the haematocrit level may lie between 35% and 55% and/or the position, given as an angle α_{FL} measured in a horizontal section of the thorax from a transverse axis A (FIG. 5), may lie between 2.9 and 3.65 rad.

Coming back to the description of the reference impedance by the surrogate model, when employing the aortic parameters, the basis functions are optionally time-independent multi-variate polynomials and the processor 8 calculates each reference impedance in step 22 from the surrogate model which describes the reference impedance by

$${f}_{i , j} ( {x}_{i} , {t}_{k} ) = {f}_{i , j} ( {x}_{i} ; {c}_{i} ( {t}_{k} ) ) = {\sum }_{q = 1 , … , Q} {c}_{i , j , q} ( {t}_{k} ) \cdot {ϕ}_{q} ( {x}_{i} ) \qquad ( 4 )$$

with
#_{q}(xᵢ) . . . a multi-variate polynomial in the aortic parameters xᵢ of the i^{th} aortic state AS₁, wherein the polynomials are indexed by q,
c_{i,j,q}(tₖ) . . . an expansion coefficient for the i^{th} aortic state ASᵢ, the j^{th} set of ICG electrodes 5_{j,in}, 5_{j,se} and the q^{th} polynomial ϕ_{q}(xᵢ) at the k^{th} sensing time tₖ,
cᵢ . . . the set of all expansion coefficients for the i^{th} aortic state ASᵢ, and

$${\sum }_{q = 1 , … , Q}$$

a sum over all Q multi-variate polynomials ϕ_{q}(xᵢ).

Optionally, the multi-variate polynomials may be chosen to be orthogonal with respect to a predetermined distribution of the aortic parameters, which yields a so-called polynomial chaos expansion.

Alternatively, other basis functions known in the art may be employed, e.g., non-polynomial basis functions such as Fourier basis functions, Gaussian basis function, etc., optionally combined with polynomial basis functions. In a further alternative embodiment the surrogate model comprises a neural network, e.g., a convolutional neural network, which describes the reference impedance and is trained on the aortic parameters as known in the art.

With reference to FIGS. 4 and 5, it shall now be described how the surrogate models are generated. This can optionally be done prior to performing the method 9, e.g., on the basis of a large number of human individuals (or animals in case the patient 2 is animal) constituting said reference objects. Therein, the impedances of each individual are measured by said sets 4ⱼ of ICG electrodes 5_{j,in}, 5_{j,se} as described above for the patient 2 and the aortic state and all aortic parameters are also examined. Alternatively, the reference objects are finite element method (FEM) models of thoraces, an example of which is shown in FIG. 5 and marked with reference number 20; in this case, the generation of the surrogate models is either carried out prior to performing the method 9, i.e., separate from the measuring system 1 as mentioned above, or by the processor 8 in step 24, in either of the two variants by simulation.

Steps 25-28 shown in FIG. 4 illustrate details of the variant in which the processor 8 generates the surrogate model, i.e., the processor 8 carries out step 24. Steps 25-27 are repeated for each of the plurality of reference objects 20 having the same aortic state, and are repeated for each aortic state to generate all surrogate models. The values of the aortic parameters of the aortic state of the reference object 20 are determined in step 25. To achieve this, the processor 8 creates the FEM model 20 (FIG. 5) with predetermined values of the aortic parameters, e.g., to cover, with the entirety of reference objects, the abovementioned range of the aortic parameters in the simulation.

In step 26, the respective set 4ⱼ of (here: simulated) ICG electrodes 5_{j,in}, 5_{j,se} are applied (in simulation) at the respective spots 14_{j,in}, 14_{j,se} on the FEM model 20 as shown in FIG. 5. As mentioned above, the spots 14_{j,in}, 14_{j,se} on the thorax 15 of the patient 2 and on the FEM thorax 20 are equivalent.

In subsequent step 27, the impedance over time is quantified by means of the (simulated) applied ICG electrodes 5_{j,in}, 5_{j,se}. This is done by simulating the injection of the current I by means of the injector electrodes 5_{j,in} and the sensing of the impedance over time by means of the sensor electrodes 5_{j,se}, e.g., by solving the Laplace equation for the electric potential in the FEM model 20 and considering the impact of blood flow through the aorta in the FEM model 20. Steps 26 and 27 are repeated for each set 4ⱼ of ICG electrodes 5_{j,in}, 5_{j,se} to obtain a number of quantified impedances for the reference object.

As illustrated in FIG. 5, the FEM model 20 can be of any desired level of detail, e.g., considering the reference object's lungs 29 and/or other organs, liquids, tissues, etc.

From the values of the aortic parameters determined in step 25 and the impedances over time quantified in step 27, the processor 8 then calculates the expansion coefficient of the respective surrogate model in step 28. Therefor, the processor 8 employs a regression or fitting method. Optionally, the processor 8 calculates the expansion coefficients of equation (4) according to

$${c}_{i , j , q} ( {t}_{k} ) = {\sum }_{l} {( {( {U}_{i}^{T} \cdot {U}_{i} )}^{- 1} \cdot {U}_{i}^{T} )}_{ql} {Z}_{j}^{l} ( {t}_{k} ) \qquad ( 5 )$$

with
Uᵢ=ϕ_{q}(vᵢ^{l}) . . . a matrix for the i^{th} aortic state ASᵢ formed by evaluating the q^{th} multi-variate polynomial at the determined values vᵢ^{l} of the aortic parameters xᵢ of the l^{th} reference object having the i^{th} aortic state ASᵢ, and
Zⱼ (tₖ) . . . the quantified impedance of the l^{th} reference object in the j set of ICG electrodes 13ⱼ at the k^{th} sensing time tₖ.

The calculated expansion coefficients, thus, parameterise the surrogate model such that the processor 8 can determine the reference impedances therefrom.

The present disclosed subject matter is not restricted to the specific embodiments and variants described in detail herein but encompasses all those variants, combinations and modifications thereof that fall within the scope of the appended claims.

What is claimed is:

1. A method for determining an aortic state of a patient by means of a processing device which has a processor and a memory connected thereto, the method comprising:
receiving in said memory, from each of at least two different sets of impedance cardiography (ICG) electrodes placed in a tetra-polar electrode configuration at respective spots on the patient's thorax, an impedance sensed over time by that set of ICG electrodes;
prior to, during, or after said receiving, storing in said memory, for each of a predetermined set of aortic states and each set of ICG electrodes, a surrogate model generated for a plurality of reference objects having that aortic state, which surrogate model describes a reference impedance over time;
computing, by means of the processor, for each aortic state of the set, a similarity value from a product of probability functions each of which depends on a difference between the sensed impedance for a respective set of ICG electrodes and the reference impedance for that aortic state and the same set of ICG electrodes; and
determining, by means of the processor, the aortic state with the highest computed similarity value as the patient's aortic state.

2. The method according to claim 1, wherein each probability function is a Gaussian function according to
$$\frac{1}{\sqrt{2 {πΔ}_{i , j , k}^{2}}} exp ( - \frac{{( {Z}_{j} ( {t}_{k} ) - {f}_{i , j} ( {t}_{k} ) )}^{2}}{2 {Δ}_{i , j , k}^{2}} )$$
with
exp . . . an exponential function,
Zⱼ(tₖ) . . . the sensed impedance for the j^{th} set of ICG electrodes at a k^{th} sensing time,
f_{i,j}(tₖ) . . . the reference impedance for the i^{th} aortic state and the j^{th} set of ICG electrodes at the k^{th} sensing time tₖ, and
Δ_{i,j,k} . . . an estimated uncertainty either as to the sensing by the j^{th} set of ICG electrodes, or as to the reference impedance f_{i,j} for the i^{th} aortic state and the j^{th} set of ICG electrodes at the k^{th} sensing time tₖ, or as to both.

3. The method according to claim 1, wherein each reference impedance is an expansion of basis functions which depend on predefined aortic parameters of the aortic state, and
wherein said step of computing is performed by marginalising the product of probability functions with respect to the aortic parameters.

4. The method according to claim 3, wherein the aortic parameters of a healthy aortic state are a haematocrit level and a maximum radius of a true lumen.

5. The method according to claim 3, wherein the aortic parameters of a dissected aortic state are a haematocrit level, a maximum radius of a true lumen, a maximum radius of a false lumen, and a position of the false lumen relative to the true lumen.

6. The method according to claim 3, wherein, in said step of computing, each similarity value is computed according to
$$p ( {AS}_{i} \vert Z ) = \frac{p ( {AS}_{i} )}{Vol ( {R}_{i} )} {\int }_{{R}_{i}} p ( Z \vert {x}_{i} , {AS}_{i} ) {dV}_{{x}_{i}}$$
with
p(ASᵢ|Z) . . . the computed similarity value of the i^{th} aortic state given sensed impedances Z,
p(ASᵢ) . . . a predetermined prior probability for the i^{th} aortic state,
Rᵢ . . . a predetermined set of values of the aortic parameters xᵢ of the i^{th} aortic state,
Vol(Rᵢ) . . . a hypervolume spanned by said predetermined set of values,
p(Z|xᵢ,ASᵢ) . . . the product of probability functions,
dVₓ_{ᵢ} . . . a hypervolume element, and
$${\int }_{{R}_{i}}$$
an integration over the predetermined set of values.

7. The method according to claim 3, wherein, in said step of computing, each reference impedance is calculated by means of the processor from the surrogate model which describes the reference impedance by
$${f}_{i , j} ( {x}_{i} , {t}_{k} ) = {f}_{i , j} ( {x}_{i} , ; {c}_{i} ( {t}_{k} ) ) = {\sum }_{q = 1 , … , Q} {c}_{i , j , q} ( {t}_{k} ) \cdot {ϕ}_{q} ( {x}_{i} )$$
with
f_{i,j}(xᵢ,tₖ) . . . the calculated reference impedance for the i^{th} aortic state and the j^{th} set of ICG electrodes at a k^{th} sensing time tₖ in dependence on the aortic parameters xᵢ of the i^{th} aortic state,
ϕ_{q}(xᵢ) . . . a multi-variate polynomial in the aortic parameters xᵢ of the i^{th} aortic state, wherein the polynomials are indexed by q,
c_{i,j,q}(tₖ) . . . an expansion coefficient for the i^{th} aortic state, the j^{th} set of ICG electrodes and the q^{th} polynomial at the k^{th} sensing time,
cᵢ . . . the set of all expansion coefficients for the i^{th} aortic state, and
$${\sum }_{q = 1 , … , Q}$$
a sum over all Q multi-variate polynomials.

8. The method according to claim 3, wherein the reference objects are finite element method (FEM) models of thoraces and the method further comprises, prior to said step of storing, generating, by means of the processor, each surrogate model by:
determining, for each of the reference objects, values of the aortic parameters of the aortic state of that reference object;
applying, for each set of ICG electrodes and each of the reference objects, the ICG electrodes on that reference object's thorax at the respective spots and quantifying, by means of the applied ICG electrodes an impedance over time; and
calculating, by means of the processor from the determined values of the aortic parameters and the quantified impedances, the expansion coefficients.

9. The method according to claim 8, wherein the expansion coefficients are calculated according to
$${c}_{i , j , q} ( {t}_{k} ) = {\sum }_{l} {( {( {U}_{i}^{T} \cdot {U}_{i} )}^{- 1} \cdot {U}_{i}^{T} )}_{q l} {Z}_{j}^{l} ( {t}_{k} )$$
with
c_{i,j,q}(tₖ) . . . the expansion coefficient for the i^{th} aortic state, the j^{th} set of ICG electrodes and a q^{th} polynomial at the k^{th} sensing time tₖ,
Uᵢ=ϕ_{q}(vᵢ^{l}) . . . a matrix for the i^{th} aortic state formed by evaluating the q^{th} multi-variate polynomial at the determined values vᵢ^{l} of the aortic parameters xᵢ of the l^{th} reference object having the i^{th} aortic state, and
Zⱼ^{l}(tₖ) . . . the quantified impedance of the l^{th} reference object in the j^{th} set of ICG electrodes at the k^{th} sensing time tₖ.

10. A method for measuring an aortic state of a patient, comprising the method according to claim 1, prior to said step of receiving, for each set of ICG electrodes:
placing the ICG electrodes of that set in the tetrapolar electrode configuration at respective spots on the patient's thorax,
sensing, by means of that set of ICG electrodes the respective impedance over time, and
transmitting, by means of that set of ICG electrodes, the sensed impedance to the memory.

11. A processing device for determining an aortic state of a patient, the processing device having
a memory which is configured to receive, from at least two different sets of impedance cardiography (ICG) electrodes placed in a tetra-polar electrode configuration at respective spots on the patient's thorax, an impedance sensed over time by that set of ICG electrodes, and to store, for each of a predetermined set of aortic states and each set of ICG electrodes, a surrogate model generated for a plurality of reference objects having that aortic state, which surrogate model describes a reference impedance over time; and
a processor connected to said memory and configured to compute, for each aortic state of the set, a similarity value from a product of probability functions each of which depends on a difference between the sensed impedance for a respective set of ICG electrodes and the reference impedance for that aortic state and the same set of ICG electrodes and to determine the aortic state with the highest computed similarity value as the patient's aortic state.

12. The processing device according to claim 11, wherein each reference impedance is an expansion of basis functions which depend on predefined aortic parameters of the aortic state, and
wherein the processor is configured to perform said step of computing by marginalising the product of probability functions with respect to the aortic parameters.

13. The processing device according to claim 12, wherein the processor is configured to compute the similarity value of each aortic state according to
$$p ( {AS}_{i} \vert Z ) = \frac{p ( {AS}_{i} )}{Vol ( {R}_{i} )} {\int }_{{R}_{i}} p ( Z \vert {x}_{i} , {AS}_{i} ) {dV}_{{x}_{i}}$$
with
p(ASᵢ|Z) . . . the computed similarity value of the i^{th} aortic state given the sensed impedances Z,
p(ASᵢ) . . . a predetermined prior probability for the i^{th} aortic state ASᵢ,
Rᵢ . . . a predetermined set of values of the aortic parameters xᵢ of the i^{th} aortic state,
Vol(Rᵢ) . . . a hypervolume spanned by said predetermined set of values,
p(Z|xᵢ,ASᵢ) . . . the product of probability functions,
dVₓ_{ᵢ} . . . a hypervolume element, and
$${\int }_{{R}_{i}}$$
an integration over the predetermined set of values.

14. The processing device according to claim 12, wherein the processor is further configured to calculate each reference impedance from the surrogate model which describes the reference impedance by
$${f}_{i , j} ( {x}_{i} , {t}_{k} ) = {f}_{i , j} ( {x}_{i} , ; {c}_{i} ( {t}_{k} ) ) = {\sum }_{q = 1 , … , Q} {c}_{i , j , q} ( {t}_{k} ) \cdot {ϕ}_{q} ( {x}_{i} )$$
with
f_{i,j}(xᵢ,tₖ) . . . the calculated reference impedance for the i^{th} aortic state and the j^{th} set of ICG electrodes at a k^{th} sensing time tₖ in dependence on the aortic parameters xᵢ of the i^{th} aortic state,
ϕ_{q}(xᵢ) . . . a multi-variate polynomial in the aortic parameters xᵢ of the i^{th} aortic state, wherein the polynomials are indexed by q,
c_{i,j,q}(tₖ) . . . an expansion coefficient for the i^{th} aortic state, the j^{th} set of ICG electrodes and the q^{th} polynomial at the k^{th} sensing time tₖ,
cᵢ . . . the set of all expansion coefficients for the i^{th} aortic state, and
$${\sum }_{q = 1 , … , Q}$$
a sum over all Q multi-variate polynomials.

15. A measuring system comprising the processing device according to claim 11 and said at least two different sets of ICG electrodes, wherein each set of ICG electrodes is configured to be placed in the tetra-polar electrode configuration at the respective spots on the patient's thorax, to sense said impedance over time and to transmit the sensed impedance to the memory.

16. The method according to claim 3, wherein, in said step of computing, each similarity value is computed according to
with
$$p ( {AS}_{i} \vert Z ) = \frac{p ( {AS}_{i} )}{Vol ( {R}_{i} )} {\int }_{{R}_{i}} {\prod }_{{j = 1 , … , J}_{k = 1 , … , K}} \frac{1}{\sqrt{2 π {Δ}_{i , j , k}^{2}}} exp ( - \frac{{( {Z}_{j} ( {t}_{k} ) - {f}_{i , j} ( {t}_{k} , {x}_{i} ) )}^{2}}{2 {Δ}_{i , j , k}^{2}} ) {dV}_{{x}_{i}}$$
with
p(ASᵢ|Z) . . . the computed similarity value of the i^{th} aortic state given the sensed impedances Z,
p(ASᵢ) . . . a predetermined prior probability for the i^{th} aortic state,
Rᵢ . . . a predetermined set of values of the aortic parameters xᵢ of the i^{th} aortic state,
Vol(Rᵢ) . . . a hypervolume spanned by said predetermined set of values,
dVₓ_{ᵢ} . . . a hypervolume element,
$${\int }_{{R}_{i}}$$
an integration over the predetermined set of values,
$${\prod }_{{j = 1 , … , J}_{k = 1 , … , K}}$$
a product over all J sets of ICG electrodes and all K sensing times,
Zⱼ(tₖ) . . . the sensed impedance for the j^{th} set of ICG electrodes at the k^{th} sensing time tₖ,
f_{i,j}(tₖ) . . . the reference impedance for the i^{th} aortic state and the j^{th} set of ICG electrodes at the k^{th} sensing time tₖ,
Δ_{i,j,k} . . . an estimated uncertainty as to the sensing by the j^{th} set of ICG electrodes and/or as to the reference impedance f_{i,j} for the i^{th} aortic state and the j^{th} set of ICG electrodes at the k^{th} sensing time tₖ, or as to both.

17. The processing device according to claim 12, wherein the processor is configured to compute the similarity value of each aortic state according to
$$p ( {AS}_{i} \vert Z ) = \frac{p ( {AS}_{i} )}{Vol ( {R}_{i} )} {\int }_{{R}_{i}} {\prod }_{{j = 1 , … , J}_{k = 1 , … , K}} \frac{1}{\sqrt{2 π {Δ}_{i , j , k}^{2}}} exp ( - \frac{{( {Z}_{j} ( {t}_{k} ) - {f}_{i , j} ( {x}_{i} , {t}_{k} ) )}^{2}}{2 {Δ}_{i , j , k}^{2}} ) {dV}_{{x}_{i}}$$
with
p(ASᵢ|Z) . . . the computed similarity value of the i^{th} aortic state given the sensed impedances Z,
p(ASᵢ) . . . a predetermined prior probability for the i^{th} aortic state ASᵢ,
Rᵢ . . . a predetermined set of values of the aortic parameters xᵢ of the i^{th} aortic state,
Vol(Rᵢ) . . . a hypervolume spanned by said predetermined set of values,
dVₓ_{ᵢ} . . . a hypervolume element,
$${\int }_{{R}_{i}}$$
an integration over the predetermined set of values,
$${\prod }_{{j = 1 , … , J}_{k = 1 , … , K}}$$
a product over all J sets of ICG electrodes and all K sensing times,
Zⱼ(tₖ) . . . the sensed impedance for the j^{th} set of ICG electrodes at the k^{th} sensing time tₖ,
f_{i,j}(xᵢ,tₖ) . . . the reference impedance for the i^{th} aortic state and the j^{th} set of ICG electrodes at the k^{th} sensing time tₖ in dependence on the aortic parameters xᵢ of the i^{th} aortic state, and
Δ_{i,j,k} . . . an estimated uncertainty as to the sensing by the j^{th} set of ICG electrodes, or as to the reference impedance f_{i,j} for the i^{th} aortic state and the j^{th} set of ICG electrodes at the k^{th} sensing time tₖ, or as to both.
